(19) United States
(12) Patent Application Publication
(10) Pub. No.: US 2024/0254556 A1
(43) Pub. Date: Aug. 1, 2024

(54) NOVEL PREDICTIVE BIOMARKERS FOR SECONDARY AUTOIMMUNITY AFTER LYMPHOCYTE DEPLETING THERAPY

(71) Applicant: GENZYME CORPORATION, Cambridge, MA (US)

(72) Inventors: Darren Phillip BAKER, Hingham, MA (US); Emanuele DE RINALDIS, Boston, MA (US); Richa HANAMSAGAR, Northborough, MA (US); Evis HAVARI, Ashland, MA (US); Virginia SAVOVA, Brookline, MA (US); Srinivas SHANKARA, Shrewsbury, MA (US)

(73) Assignee: GENZYME CORPORATION, Cambridge, MA (US)

(21) Appl. No.: 18/560,569

(22) PCT Filed: May 13, 2022
(86) PCT No.: PCT/US2022/029262

§ 371 Date: Nov. 13, 2023

Related U.S. Application Data
(60) Provisional application No. 63/188,302, filed on May 13, 2021.

(51) Int. Cl. C12Q 1/6883; C12Q 1/6874; G01N 33/564

(52) U.S. Cl. CPC C12Q 1/6883; C12Q 1/6874; G01N 33/564; C12Q 2600/158; G01N 2800/046; G01N 2800/285; G01N 2800/50; G01N 2800/52

(57) ABSTRACT

The invention provides methods of assessing the risk of secondary autoimmunity in a patient with a primary autoimmune disease (e.g., MS) following lymphocyte depleting therapy (e.g., anti-CD52 antibody therapy) based on the fraction of a novel cell type termed platelet lineage cells (PLCs) among total cells, and/or the Immature Platelet Fraction (IPF) value, in a biological sample from the patient.

### CROSS-REFERENCE TO RELATED APPLICATIONS

This application claims priority from U.S. Provisional Patent Application 63/188,302, filed May 13, 2021. The disclosure of that priority application is incorporated by reference in its entirety herein.

### BACKGROUND OF THE INVENTION

Multiple sclerosis (MS) is a chronic, immune-mediated inflammatory and neurodegenerative disease that affects the central nervous system. It is characterized by loss of motor and sensory function resulting from inflammation, demyelination, and axonal injury and loss (Friese et al., Nat Rev Neurol. (2014) 10(4):225-38; Trapp and Nave, Ann Rev Neurosci. (2008) 231:247-69). MS patients display a wide range of severe clinical symptoms with increased physical disability, fatigue, pain, and cognitive impairment as the disease progresses. MS affects more than two million people worldwide and is at least two to three times more prevalent in women than in men. It has a significant impact on patients' quality of life and shortens patients' life expectancy by five to ten years on average.

Alemtuzumab, a humanized anti-CD52 monoclonal antibody, is an approved treatment for relapsing forms of MS (RMS). While its efficacy has been demonstrated in clinical studies, its use is associated with unpredictable non-MS secondary autoimmunity manifesting months or years after treatment. Approximately 40% of alemtuzumab treated patients present with autoimmune thyroid events, 2% with platelet deficiency (immune thrombocytopenia; ITP) (Cuker et al., Mult Scler Houndmills Basingstoke Engl. (2020) 26:48-56), and 0.34% with autoimmune nephropathies (Phelps et al., Mult Scler Houndmills Basingstoke Engl. (2019) 25:1273-88). The lack of predictive biomarkers for secondary autoimmunity necessitates careful monitoring in clinical practice, with a Risk Management Plan or Risk Evaluation and Mitigation Strategy (RMP/REMS) in place for early detection of these autoimmune events.

Thus, there is an unmet need for identifying novel biomarkers for predicting the risk of development of secondary autoimmunity in patients considering treatment with a lymphocyte depleting therapy such as alemtuzumab.

### SUMMARY OF THE INVENTION

The present disclosure provides new and useful methods for improving risk management in treatment of autoimmune diseases such as MS. The methods reduce treatment side effects such as secondary autoimmunity, and help health care providers and patients in selecting regimens for autoimmune disease treatment and post-treatment monitoring. The methods of the present disclosure are based on the discovery that in MS patients, low abundance of platelet lineage cells (PLCs) and/or high immature platelet fraction (IPF) values detected even before lymphocyte depleting therapy (e.g., alemtuzumab therapy) correlate with increased risk of developing secondary autoimmunity after the therapy.

In one aspect, the present disclosure provides a method for assessing the risk of developing secondary autoimmunity in a patient with a primary autoimmune disease following lymphocyte depleting therapy, comprising:
a) providing a blood sample from the patient; and
b) determining
   (i) the fraction of platelet lineage cells (PLCs) (e.g., mature PLCs) among the total cells in the blood sample, wherein a reduced fraction of PLCs (e.g., mature PLCs) compared to a first reference is indicative of a heightened risk of developing secondary autoimmunity in the patient after treatment, and/or
   (ii) the Immature Platelet Fraction (IPF) in the blood sample, wherein an increased IPF compared to a second reference is indicative of a heightened risk of developing secondary autoimmunity in the patient after treatment.

In certain embodiments, the method comprises determining both (i) and (ii).

In another aspect, the present disclosure provides a method for treating a patient with a primary autoimmune disease, comprising:
a) selecting a patient who has been diagnosed as not being at a heightened risk of developing secondary autoimmunity after lymphocyte depleting therapy, wherein the risk has been diagnosed by determining
   (i) the fraction of platelet lineage cells (PLCs) (e.g., mature PLCs) among the total cells in the blood sample, wherein a reduced fraction of PLCs (e.g., mature PLCs) compared to a first reference is indicative of a heightened risk of developing secondary autoimmunity in the patient after treatment, and/or
   (ii) the Immature Platelet Fraction (IPF) in the blood sample, wherein an increased IPF compared to a second reference is indicative of a heightened risk of developing secondary autoimmunity in the patient after treatment; and
b) administering a therapeutically effective amount of the lymphocyte depleting therapy to the patient.

In certain embodiments, the method comprises determining both (i) and (ii).

In some embodiments, the primary autoimmune disease is multiple sclerosis (MS). In particular embodiments, the primary autoimmune disease is relapsing MS, relapsing-remitting MS (RR-MS), or secondary progressive MS (SPMS).

In some embodiments, the lymphocyte depleting therapy is a lymphocyte depleting antibody therapy, such as an anti-CD52 antibody or an antigen-binding portion thereof. In certain embodiments, the anti-CD52 antibody has the six CDRs of alemtuzumab. In certain embodiments, the anti-CD52 antibody has the heavy and light chain variable domains of alemtuzumab. In particular embodiments, the anti-CD52 antibody is alemtuzumab.

In some embodiments of the methods described herein, the first and second references are obtained from a patient with said primary autoimmune disease who does not develop secondary autoimmunity after lymphocyte depleting treatment, or from a healthy subject.

In some embodiments, the blood sample is an erythrocyte-lysed blood sample. In some embodiments, the blood sample is a peripheral blood monocyte cell (PBMC) sample (e.g., wherein neutrophils in the sample have been removed).

In some embodiments, the PLC fraction is reduced by >2 standard deviations compared to that of a control subject. In some embodiments, the IPF value is increased by >2 standard deviations compared to that of a control subject.

In some embodiments, the PLCs are characterized by being CD41⁺CD61⁺SPARC⁺TREML1⁺.

The methods of the present disclosure may further comprise the step of determining the fraction of immature PLCs among the total PLC population in the biological sample from the patient, wherein an increased fraction of immature PLCs compared to a third reference is indicative of a heightened risk of developing secondary autoimmunity in the patient after treatment. In some embodiments, the immature PLCs are characterized by being CD41^{low}CD61^{low}PDGF A^{high}PDCD10^{high}, optionally further by being DAB2^{high}RGS10^{high}RGS18^{high}TSC22D1^{high}. In some embodiments, the third reference is obtained from a patient with said primary autoimmune disease who does not develop secondary autoimmunity after lymphocyte depleting treatment, or from a healthy subject.

In some embodiments, the secondary autoimmunity is selected from the group consisting of immune thrombocytopenia purpura (ITP), Graves' disease, Hashimoto's disease, Goodpasture's disease (antiglomerular basement membrane (GBM) disease), membranous glomerulonephritis (membranous nephropathy), red cell aplasia, autoimmune thyroid disease, transient thyroiditis, autoimmune hemolytic anemia, diabetes mellitus type 1, alopecia areata/alopecia totalis, vitiligo, myalgia, sarcoidosis, autoimmune neutropenia, autoimmune hepatitis, and autoimmune lymphopenia.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE DRAWINGS

FIG. 1 depicts a bar graph showing relative abundance of all major immune cell types (T cells, monocytes, B cells, and NK cells) as well as rare cell-types (plasmacytoid dendritic cells (pDCs) and platelet-like cells), and scatter bar plots showing that platelet-like cells are significantly enriched in patients who do not develop secondary autoimmunity compared to patients who develop secondary autoimmunity (Mean±S.E.M; Student's t-test **p<0.01). Solid circles represent samples collected from patients who develop secondary autoimmunity; solid squares represent samples collected from patients who do not develop secondary autoimmunity.

FIG. 2 is a scatter bar plot showing PLC abundance in individual patients based on sc-RNAseq data before and after alemtuzumab treatment in patients who developed secondary autoimmunity (sAI) and patients who did not develop secondary autoimmunity (non-sAI). The non-sAI patient represented by large squares showed reduced PLC numbers at T24 (24 months after first course), and developed sAI between 4 and 7 years of follow up.

FIG. 3 is a scatter bar plot showing that platelet-lineage cells (PLCs) are enriched in patients who do not develop secondary autoimmunity at both T0 (pre-treatment) and T24 (24 months after first alemtuzumab course) timepoints (Mean±S.E.M; Two-way ANOVA, overall effect of AI state **p<0.01). Solid circles represent samples collected from patients who develop secondary autoimmunity; solid squares represent samples collected from patients who do not develop secondary autoimmunity.

FIG. 4 depicts scatter plots and accompanying SPRING plots showing the gene expression levels of SPARC, TREML1, GP9, ITGB3, ITGA2B, and GPIB (Mean±S.E.M; Two-way ANOVA with Sidak's multiple comparison test *p<0.05, **p<0.001 and ***p<0.001). Solid circles represent samples collected from patients who develop secondary autoimmunity; solid squares represent samples collected from patients who do not develop secondary autoimmunity.

FIG. 5 is a bar graph showing the percentage of platelets in whole blood, fresh PBMCs, and frozen PBMCs by flow cytometry (Mean±S.E.M).

FIG. 6 is a bar graph showing the percentage of PLCs in whole blood, fresh PBMCs, and frozen PBMCs by flow cytometry (Mean±S.E.M).

FIG. 7 is a bar graph quantifying the percentage of PLCs in RBC-lysed blood, fresh PBMCs, and frozen PBMCs among cells that are 1) TREML1^{hi} SPARC⁺ or 2) TREML1^{lo} SPARC⁺.

FIG. 8 depicts a bar graph showing the percentage of cells within the sAI and non-sAI groups that belong to Subset 1 (immature/resting transcriptomic state) or Subset 2 (mature/activated transcriptomic state).

FIGS. 9A and 9B are heatmaps showing unsupervised clustering analysis of RNAseq data from 161 baseline samples from MS patients prior to alemtuzumab treatment. FIG. 9A depicts relative expression levels of six mature PLC genes (GPIBA, PPBP, ITGA2B, ITGB3, SPARC, and TREML1) and five immature PLC genes (PDCD10, RGS10, DAB2, TSC22D1, and RGS18) in patients who developed secondary autoimmunity (“AI-enriched,” left) and patients who did not develop secondary autoimmunity (“nonAI-enriched,” right). FIG. 9B shows the same heatmap as FIG. 9A, and further provides information at the bottom of the heatmap regarding patient traits such as thyroid activity, race, and gender.

FIGS. 10A and 10B are plots showing the expression levels of the specific genes shown in FIGS. 9A and 9B at different sampling times (0 months, 12 months, and 24 months), with the mature PLC genes shown in FIG. 10A and the immature PLC genes shown in FIG. 10B. Data is shown for patients who developed secondary autoimmunity (“AI”; top) and who did not develop secondary autoimmunity (“NonAI”; bottom).

FIG. 11 is a heatmap showing unsupervised clustering analysis of RNAseq data from MS patient baseline samples, wherein some of the patients were treated with IFN beta-1a. Data is provided for the same mature and immature PLC genes shown in FIGS. 9A and 9B. “AI”: patients who developed secondary autoimmunity. “NonAI”: patients who did not develop secondary autoimmunity.

FIG. 12A is a box and whisker plot of immature platelet fraction (IPF) clinical values at T0 (baseline) in patients who develop secondary autoimmunity (sAI; solid circles) and patients who do not develop secondary autoimmunity (non-sAI; solid squares); normal range is shown in red bracket (error bars span 10-90th percentile range).

FIG. 12B is a plot showing the change in IPF clinical values over time (0-24 months) in patients who develop secondary autoimmunity (sAI; solid circles) and patients who do not develop secondary autoimmunity (non-sAI; solid squares) (Mean±S.E.M.; Two-way ANOVA, overall sAI state difference ***p=0.0001).

FIG. 13 is a correlation graph of IPF clinical values and percentage of PLCs from single-cell data for patients who develop secondary autoimmunity (sAI; solid circles) and patients who do not develop secondary autoimmunity (non-sAI; solid squares) at TO (baseline). Left: Post-hoc analysis of true sAI and true non-sAI identification of patients within the current cohort, based on either IPF values alone or IPF and percentage of PLC values together. Right: Tabular depiction of combinatorial use of clinical IPF values and single-cell PLC data in identifying AI status prior to treatment in the given cohort of patients.

### DETAILED DESCRIPTION OF THE INVENTION

The present disclosure is based on the discovery that the occurrence of secondary autoimmunity in a patient with a primary autoimmune disease (e.g., MS) following lymphocyte depleting therapy is associated with a low abundance of platelet lineage cells (PLCs) and/or high immature platelet fraction (IPF) values compared to a control subject, even before lymphocyte depletion. The control subject may be, e.g., a healthy subject or a patient with a primary autoimmune disease who does not develop secondary autoimmunity after lymphocyte-depleting therapy. Thus, a reduced fraction of PLCs and/or an increased IPF are predictive biomarkers for assessing the risk of occurrence of secondary autoimmunity following lymphocyte depletion.

Based on the above findings, the present disclosure provides methods for improving risk management of patients with a primary autoimmune disease (e.g., MS) when considering lymphocyte depleting therapy such as therapy with an anti-CD52 antibody (e.g., alemtuzumab). For example, by allowing a health care provider to determine whether an MS patient is or is not at an increased risk of developing secondary autoimmunity following the therapy, the health care provider can determine whether the patient should undergo the therapy (e.g., if the patient is not at increased risk), or whether the patient should not undergo the therapy or have heightened monitoring for secondary autoimmunity after the therapy (e.g., if the patient is at heightened risk).

For example, the present disclosure provides methods for assessing the risk of developing secondary autoimmunity in a patient with a primary autoimmune disease (e.g., MS) who is at increased risk of developing a secondary autoimmune disease following lymphocyte depleting therapy. In some embodiments, a patient assessed as not being at increased risk is treated with the lymphocyte depleting therapy. In some embodiments, a patient assessed as being at increased risk is not treated with the lymphocyte depleting therapy. In some embodiments, a patient assessed as being at increased risk is treated with the lymphocyte depleting therapy and then receives heightened monitoring in comparison to patients identified as not being at increased risk.

The present disclosure also provides methods for treating a patient with a primary autoimmune disease (e.g., an MS patient) who is not at increased risk of developing a secondary autoimmune disease following lymphocyte depleting therapy.

The present disclosure also provides methods for treating a patient with a primary autoimmune disease (e.g., an MS patient) who is at increased risk of developing a secondary autoimmune disease following lymphocyte depleting therapy, wherein the therapy is followed by heightened monitoring for developing secondary autoimmunity (compared to monitoring for patients not at increased risk). Such heightened monitoring can follow an appropriate monitoring regimen determined by the health care provider following lymphocyte depleting therapy. An appropriate monitoring regimen for patients at risk may include, without limitation, more frequent monitoring for secondary autoimmunity after lymphocyte depleting therapy at an interval of, for example, one week, two weeks, one month, two months, three months, six months, or one year. The monitoring may be continued for an extended period of time, for example, more than one year, two years, three years, four years, five years, or more, because some patients may not present with secondary autoimmunity until well after one year following lymphocyte depletion therapy. Heightened monitoring also may entail, for example, more thorough medical examination (e.g., more blood tests) by a specialist for any signs of secondary autoimmunity. Moreover, pharmacists or clinical staff who distribute a lymphocyte depleting drug to a patient for treating MS may be required to counsel the patient on the increased risk of developing secondary autoimmunity following the drug use, in the event that the patient has reduced PLC levels and/or an elevated IPF value (optionally with elevated levels of immature PLCs). The pharmacists or clinical staff may also be required to obtain informed consent from the patient prior to distributing the drug to the patient.

### I. Methods of Risk Assessment

The risk to an autoimmune disease (e.g., MS) patient of developing secondary autoimmunity after lymphocyte depletion may be assessed by determining:
i) the fraction of platelet lineage cells (PLCs) (e.g., mature PLCs) among the total cells in a biological (e.g., blood) sample from the patient, wherein a reduced fraction of PLCs (e.g., mature PLCs) compared to a control subject is indicative of a heightened risk; and/or
ii) the Immature Platelet Fraction (IPF) in the biological sample, wherein an increased IPF compared to a control subject is indicative of a heightened risk.

In some embodiments, the risk is assessed by determining i) (and optionally ii)) and also iii) the fraction of immature PLCs among the total PLC population in the biological sample, wherein an increased fraction of immature PLCs compared to a control subject is indicative of a heightened risk. In some embodiments, the risk is assessed by determining iii), or ii) and iii).

In some embodiments, the risk is assessed by determining i), ii), iii), or any combination thereof, and also iv) testing for the presence of antibodies against mature or activated platelets in the biological sample, wherein increased anti-(mature/activated) platelet antibodies compared to a control subject are indicative of a heightened risk. In some embodiments, the risk is assessed by determining iv).

In certain embodiments, the biological sample obtained from the patient is a body fluid sample such as blood (e.g., whole blood, freshly isolated peripheral blood mononuclear cells (PBMCs), or frozen PBMCs), serum, plasma, urine, saliva, lymphatic fluid, or cerebrospinal fluid. In particular embodiments, the biological sample is blood that is erythrocyte (RNA)-lysed.

In certain embodiments, relative PLC abundance, IPF value, and/or immature PLC fraction in a patient who develops post-treatment secondary autoimmunity are in reference to a control subject, e.g., a healthy subject. The healthy subject, in this context, is an individual without any known inflammatory condition, including without an autoimmune disease (e.g., without any detectable symptoms of an autoimmune disease). In some embodiments, the healthy subject is not lymphopenic. In some embodiments, the control subject is an autoimmune disease patient who does not develop secondary autoimmunity after lymphocyte depletion.

Obtaining information on the relative PLC abundance, the IPF value, and/or the immature PLC fraction in a biological sample from an autoimmune disease (e.g., MS) patient is useful in selecting treatment and post-treatment monitoring regimens for the patient. When the information is obtained prior to lymphocyte depleting therapy, the patient can be informed of the relative risk of developing secondary autoimmunity following therapy and treatment decisions can be made accordingly. The patient also can be informed of a need for heightened post-treatment monitoring, e.g., more frequent and more thorough examination by a specialist, if he/she is classified as “at risk.” Thus, this information improves risk management (by physicians, pharmacists, and patients) in treatment of autoimmune disease. Obtaining the information during or after lymphocyte depletion treatment also may be helpful in monitoring secondary autoimmunity development and determining treatment.

### A. Platelet Lineage Cells

Platelet lineage cells (PLCs) are a novel, rare platelet-like cell-type that strongly resembles platelets, but differs from platelets in its larger size, granularity, and transcript content. Besides expressing classical platelet markers like CD41 and CD61, PLCs also express additional surface markers, including SPARC (Secreted Protein Acidic and Rich in Cysteine) and TREML1 (Triggering Receptor Expressed on Myeloid Cells Like 1), that are not ubiquitously associated with platelets at high levels.

As detailed below in the Examples, PLCs were found to comprise two distinct subsets that differ in their expression of several markers. The first subset (Subset 1) is characterized by lower expression of platelet markers, and higher expression of platelet derived growth factor subunit A (PDGFA), inhibitory markers (e.g., programmed cell death 10 (PDCD10)), and nuclear proteins (e.g., DAB2, RGS10, RGS18, and TSC22D1). The second subset (Subset 2) is relatively higher in the expression of actin genes ACTB and ACTG1, and PPBP, a platelet derived growth factor that is a potent chemoattractant and activator of neutrophils. The second subset is also enriched in SPARC and TREML1 gene expression. Subset 1 encompasses most PLCs in patients who develop post-treatment secondary autoimmunity. The inventors have further discovered that there is a difference in maturity and activation state between the two subsets, with Subset 1 representing immature or resting state PLCs (enriched in patients who present with post-treatment secondary autoimmunity), while Subset 2 represents mature or activated PLCs.

Thus, PLCs may be identified based on the expression of specific cell surface markers. In some embodiments, PLCs may be identified based on the concurrent expression of CD41, CD61, SPARC, and/or TREML1 (e.g., CD41 CD61 SPARC-TREML1″). In certain embodiments, mature or activated PLCs may be identified based on the concurrent expression of any combination of MYL9, CLU, PPBP, SPARC, TREML1, ACTB, NCOA4, TMSB4X, AP001189.4, F13A1, PARVB, ALOX12, RBPMS2, PVALB, PF4V1, ARPC1B, SH3BGRL3, PKM, TAGLN2, TGFB111, HLA.E, FERMT3, LTBP1, GSN, CD9, C6orf25, ITGA2B, SERF2, and C19orf33. In particular embodiments, mature or activated PLCs are identified based on the concurrent expression of GPIBA, ITGA2B, ITGB3, ACTB, ACTG1, PPBP, SPARC, and/or TREML1, such as a combination of any two, three, four, five, six, seven, or all eight of said markers (e.g., ACTB^{high} ACTG1^{high}PPBP^{high}SPARC^{high}TREML1^{high}). In certain embodiments, immature or resting PLCs may be identified based on the concurrent expression of any combination of RGS18, ACRBP, PTCRA, TSC22D1, HIST1H3H, HIST1H2AC, MYL4, HIST1H2BJ, TMEM40, SLC40A1, SMIM5, TALI, PEGFA, FAM110A, THEM5, ARHGAP6, NFE2, MMD, NEXN, SCGBIC1, DNM3, GP6, GFIIB, LIMS1, GSTO1, DAB2, ERV3.1, ELOVL7, and LCN2. In particular embodiments, immature or resting PLCs may be identified based on the concurrent expression of PDGFA, PDCD10, DAB2, RGS10, RGS18, and/or TSC22D1, such as a combination of any two, three, four, five, or all six of said markers (e.g., DAB2^{high}RGS10^{high}RGS18^{high}TSC22D1^{high}). For example, immature PLCs may be characterized as being CD41^{low}CD61^{low}PDGF A^{high}PDCD10^{high}

In the methods of the present disclosure, relative PLC abundance (the fraction of PLCs, e.g., mature PLCs, among total cells) can be measured by a number of techniques well known to those skilled in the art. In some embodiments, a biological sample is obtained from a subject, and relative PLC abundance in the sample is measured by any method or assay suitable for detection of RNA-containing cells. In certain embodiments, relative PLC abundance is measured by using flow cytometry analysis (e.g., high dimensional flow cytometry analysis), such as a fluorescence-activated cell sorting (FACS) assay, or by nCounterR. In certain other embodiments, relative PLC abundance is measured using single-cell RNA sequencing (scRNA-seq). In specific embodiments, the scRNA-seq is droplet-based parallel scRNA-seq.

In some embodiments, an autoimmune disease (e.g., MS) patient at increased risk for developing secondary autoimmunity following lymphocyte depleting therapy (e.g., an anti-CD52 antibody therapy such as alemtuzumab) has a reduced fraction of PLCs among total cells in a biological sample (e.g., blood) compared to a control subject, wherein the PLC fraction is reduced by >1.5, >2, >3, >4, or >5 (e.g., >2) standard deviations compared to that of a control subject.

In some embodiments, an autoimmune disease (e.g., MS) patient at increased risk for developing secondary autoimmunity following lymphocyte depleting therapy (e.g., an anti-CD52 antibody therapy such as alemtuzumab) has an increased fraction of immature PLCs among total PLCs in a biological sample (e.g., blood) compared to a control subject, wherein the immature PLC fraction is increased by >1.5, >2, >3, >4, or >5 (e.g., >2) standard deviations compared to that of a control subject.

Certain statistical analyses can be applied to determine if the relative PLC abundance or immature PLC fraction in a test sample is significantly different from a reference level (e.g., from a control subject). Such statistical analyses are well known to those skilled in the art and may include, without limitation, parametric (e.g., two-tailed Student's t-test) or non-parametric (e.g., Wilcoxon-Mann-Whitney U test) tests.

### B. Immature Platelet Fraction (IPF)

Autoimmune disease patients who develop secondary autoimmunity after lymphocyte depleting treatment may differ in clinical measures of platelet maturity from patients who do not develop post-treatment secondary autoimmunity. Specifically, the inventors have discovered that patients who develop thyroid autoimmunity post-alemtuzumab treatment have significantly higher pre-treatment Immature Platelet Fraction (IPF) values compared to those who do not develop thyroid autoimmunity.

IPF reflects the fraction of circulating platelets which still retain RNA. It is a parameter measuring young, reticulated, platelets in peripheral blood. The IPF is usually high in conditions where rapid platelet destruction is observed.

In the methods of the present disclosure, IPF can be measured by a number of techniques well known to those skilled in the art. IPF is usually determined by flow cytometry (e.g., high dimensional flow cytometry) or hematology analysis. For instance, the residual RNA content of immature platelets can readily be stained with dyes such as thiazole orange (TO) and IPF can be measured using flow cytometry. Alternatively, IPF may be quantified using an optical fluorescence method conducted in the reticulocyte/optical platelet channel of an automated hematology system. In this approach, a polymethine fluorescent dye is used to stain the RNA/DNA of the reticulated cells, platelet membranes, and granules. This method allows the simultaneous counting of reticulocytes, erythrocytes, and fluorescent platelets. Other methods of quantifying IPF may include, e.g., looking for specific transcriptomic signatures of IPFs in a biological (e.g., peripheral blood) sample, and/or IPF enriched cell populations in a biological (e.g., whole blood) sample; or nCounterR.

Thus, in some embodiments, any method described herein for assessing an autoimmune disease (e.g., MS) patient's risk of developing secondary autoimmunity after lymphocyte depleting therapy may include a step of determining the IPF value in a biological sample from the patient. In some embodiments, a patient at increased risk for developing secondary autoimmunity following lymphocyte depleting therapy (e.g., an anti-CD52 antibody therapy such as alemtuzumab) has an increased IPF value in a biological sample (e.g., blood), wherein the IPF value is increased by >1.5, >2, >3, >4, or >5 (e.g., >2) standard deviations compared to that of a control subject.

In some embodiments, an increased risk correlates with an increased IPF value and lower PLC abundance, in comparison to a control subject.

### II. Lymphocyte Depleting Therapy

As used herein, “lymphocyte depleting therapy” refers to a type of immunosuppression by therapeutic reduction of circulating lymphocytes, e.g., T cells and/or B cells, resulting in lymphopenia. Prolonged lymphocyte depletion is seen when, e.g., autologous bone marrow transplantation (BMT) or total lymphoid irradiation is used to treat multiple sclerosis. See, e.g., Cox et al., Eur J Immunol. (2005) 35:3332-42. For example, lymphocyte depletion can be achieved by a combined use of thymoglobulin, cyclophosphamide, and whole body irradiation. Lymphocyte depletion in MS patients can also be achieved by a number of drug treatments. For example, a humanized anti-CD52 monoclonal antibody, alemtuzumab (CAMPATH-1H), has been used in lymphocyte depleting therapy to treat MS patients. Alemtuzumab-induced lymphopenia has been shown to effectively reduce central nervous system inflammation both clinically and radiologically (Coles et al., Ann. Neurol. (1999) 46:296-304; Coles et al., N. Engl. J. Med. (2008) 359:1786-1801).

Thus, in some embodiments, a lymphocyte depleting therapy described herein is an agent that targets CD52-expressing cells. In certain embodiments, the lymphocyte depleting therapy is an anti-CD52 antibody or an antigen-binding portion thereof. The antibody may be, e.g., monoclonal, polyclonal, oligoclonal, or bifunctional. In particular embodiments, the anti-CD52 antibody or antigen-binding portion binds to the same epitope as alemtuzumab. The antibody or antigen-binding portion may comprise the six CDR amino acid sequences or the heavy and light chain variable domain amino acid sequences of alemtuzumab. In certain embodiments, the anti-CD52 antibody is alemtuzumab.

The term “antigen-binding portion” as used herein refers to one or more fragments of an antibody that retain the ability to specifically bind to the same antigen as the whole antibody from which the portion is derived. Examples of “antigen-binding portion” include, without limitation, a Fab fragment, a F(ab′)2 fragment, a Fd fragment, a Fv fragment, a dAb fragment, an isolated complementarity determining region (CDR), scFv, and a diabody. The antibodies and antigen-binding portions thereof described herein can be made by any methods well known in the art.

Other agents can also be used in lymphocyte-targeting therapy to treat autoimmune disease (e.g., MS) patients. These agents can be those that cause lymphocyte cell death or inhibit lymphocyte functions. They include, without limitation, (1) agents targeting CD52-bearing cells, such as agents biologically similar to alemtuzumab, i.e., other anti-CD52 antibodies (e.g., chimeric, humanized, or human antibodies) that bind to the same or a different epitope as alemtuzumab or compete with alemtuzumab for binding to CD52; (2) biomolecules such as peptides, proteins, and antibodies (e.g., chimeric, humanized, or human antibodies) that target cell-surface molecules on lymphocytes, such as anti-CD2 antibodies, anti-CD3 antibodies, anti-CD4 antibodies, anti-CD20 antibodies (e.g., rituximab), anti-CD38 antibodies, anti-TCR antibodies, and anti-integrin antibodies (e.g., natalizumab); (3) cytotoxins (e.g., apoptosis-inducing agents, cyclophosphamide, alkylating agents, and DNA intercalators) delivered specifically or nonspecifically to lymphocytes; (4) antigen-binding portions of the aforementioned antibodies, (5) IMiDs (e.g., teriflunomide), and (6) BTK inhibitors (e.g., tolebrutinib).

### III. Patient Populations

The methods of the present disclosure can be used in the context of a patient with an autoimmune disease (“primary” autoimmune disease, to distinguish from secondary autoimmunity). The primary autoimmune disease may be, for example, multiple sclerosis (MS), N-methyl-D-aspartate receptor (NMDAR) encephalitis, scleroderma, myasthenia gravis, systemic lupus erythematosus (SLE), rheumatoid arthritis (RA), myelin-oligodendrocyte glycoprotein (MOG) spectrum disorder (MOGSD), or neuromyelitis optica spectrum disorder (NMOSD).

In some embodiments, the primary autoimmune disease is MS, e.g., relapsing-remitting MS, primary progressive MS, or secondary progressive MS. MS patients in the context of the present disclosure are those who have been diagnosed as having a form of MS by, for example, the history of symptoms and neurological examination with the help of tests such as magnetic resonance imaging (MRI), spinal taps, evoked potential tests, and laboratory analysis of blood samples.

MS, also known as disseminated sclerosis, is a complex disease characterized by considerable heterogeneity in its clinical, pathological, and radiological presentation. It is an autoimmune condition in which the immune system attacks the central nervous system, leading to demyelination (Compston and Coles, Lancet (2008) 372(9648): 1502-17). MS destroys a fatty layer called the myelin sheath that wraps around and electrically insulates nerve fibers. Almost any neurological symptom can appear with the disease, which often progresses to physical and cognitive disability (Compston and Coles, 2008). New symptoms can occur in discrete attacks (relapsing forms), or slowly accumulate over time (progressive forms) (Lublin et al., Neurology (1996) 46(4):907-11). Between attacks, symptoms may go away completely (remission), but permanent neurological problems often occur, especially as the disease advances (Lublin et al., 1996). Several subtypes, or patterns of progression, have been described, and they are important for prognosis as well as therapeutic decisions. In 1996, the United States National Multiple Sclerosis Society standardized four subtype definitions: relapsing-remitting, secondary progressive, primary progressive, and progressive relapsing (Lublin et al., 1996).

The relapsing-remitting subtype (RRMS) is characterized by unpredictable acute attacks, called exacerbations or relapses, followed by periods of months to years of relative quiet (remission) with no new signs of disease activity. This describes the initial course of most individuals with MS. RRMS is the most heterogeneous and complex phenotype of the disease, characterized by different levels of disease activity and severity, particularly in the early stages. Inflammation is predominant but there is also neurodegeneration. Demyelination occurs during acute relapses lasting days to months, followed by partial or complete recovery during periods of remission where there is no disease activity. RRMS affects about 65-70% of the MS population and tends to progress to secondary progressive MS.

Secondary progressive MS (SPMS) begins with a relapsing-remitting course, but subsequently evolves into progressive neurologic decline between acute attacks without any definite periods of remission, even though occasional relapses, minor remissions or plateaus may appear. Prior to the availability of the approved disease-modifying therapies, data from natural history studies of MS demonstrated that half of RRMS patients would transition to SPMS within 10 years and 90% within 25 years. SPMS affects approximately 20-25% of all people with MS.

The primary progressive subtype (PPMS) is characterized by a gradual but steady progression of disability with no obvious remission after the initial MS symptoms appear (Miller et al., Lancet Neurol. (2007) 6(10):903-12). It is characterized by progression of disability from onset, with occasional temporary minor improvements or plateaus. A small percentage of PPMS patients may experience relapses. Approximately 10% of all individuals with MS have PPMS. The age of onset for the primary progressive subtype is usually later than other subtypes (Miller et al., 2007). Males and females are equally affected.

Progressive relapsing MS (PRMS) is characterized by a steady neurological decline with acute attacks that may or may not be followed by some recovery. This is the least common of all the subtypes described hereinabove.

Cases with non-standard behavior have also been described, sometimes referred to as borderline forms of MS (Fontaine, Rev Neurol. (Paris) (2001) 157 (8-9 Pt 2):929-34). These forms include Devic's disease, Balo concentric sclerosis, Schilder's diffuse sclerosis, and Marburg multiple sclerosis (Capello et al., Neurol Sci. 25 Suppl (2004) 4:S361-3; Hainfellner et al., J Neurol Neurosurg Psychiatr. (1992) 55(12): 1194-6).

The regulatory phrase “relapsing forms of MS” (RMS) generally encompasses both RRMS and SPMS with relapses. The phrase generally refers to three different patient subtypes: RRMS, SPMS with relapses, and a clinically isolated demyelination event with evidence of dissemination of lesions in time and space on the MRI (see, e.g., European Medicines Agency, Committee for Medicinal Products for Human Use's “Guideline on Clinical Investigation of Medicinal Products for the Treatment of Multiple Sclerosis” (Rev. 2, 2015)).

### IV. Secondary Autoimmunity

Autoimmunity is referred to herein as “secondary autoimmunity” (sAI) when it arises subsequent to the onset of a first (“primary”) disease, for example, a “primary” autoimmune disease, e.g., MS. Secondary autoimmunity sometimes arises in MS patients having, or having had, lymphopenia following, e.g., lymphocyte depleting therapy. In some individuals, secondary autoimmunity arises soon after lymphocyte depleting therapy (e.g., treatment with alemtuzumab). In other individuals, secondary autoimmunity may not arise until months or years after lymphocyte depleting therapy; in some of those individuals, by the time they develop secondary immunity, substantial lymphocyte recovery (total lymphocyte count) may have occurred so that they may no longer be lymphopenic. Thus, in some cases, patients who have been treated with lymphocyte depleting therapy, e.g., anti-CD52 antibody, should be carefully monitored for signs of any secondary autoimmunity and timely treated.

Secondary autoimmunity includes, but is not limited to, autoimmune thyroid disease (including Grave's disease, hyperthyroidism, hypothyroidism, goiter, Hashimoto's disease, and thyroiditis (e.g., transient thyroiditis)), autoimmune cytopenias (including idiopathic thrombocytopenia purpura (ITP), autoimmune neutropenia, autoimmune hemolytic anemia, autoimmune lymphopenia, and red cell aplasia), diabetes mellitus type 1, alopecia areata (e.g., alopecia totalis), vitiligo, myalgia, sarcoidosis, autoimmune hepatitis, and nephropathies including glomerulonephritis (e.g., membranous glomerulonephritis) and antiglomerular basement membrane (GBM) disease (Goodpasture's syndrome). Techniques for diagnosing and monitoring these secondary autoimmune diseases are well known to those skilled in the art, including assessment of symptoms and medical examination such as blood analysis. The invention contemplates the use of any known methods. For example, autoantibody levels in a patient's body fluid (e.g., blood) can be determined as a means of detecting signs of secondary autoimmunity. Specifically, anti-nuclear antibodies, anti-smooth muscle antibodies, and anti-mitochondrial antibodies can be measured. In the event that anti-nuclear antibodies are detected, additional assays can be performed to measure anti-double-stranded DNA antibodies, anti-ribonucleoprotein antibodies, and anti-La antibodies. Anti-thyroid peroxidase (TPO) and anti-thyroid stimulating hormone (TSH) receptor antibodies can be measured to detect autoimmune thyroid diseases; if anti-TPO or anti-TSH receptor antibodies are detected, one can measure whether thyroid function is affected by measuring free T3, free T4 and TSH levels. Anti-platelet antibodies can be measured to detect autoimmune thrombocytopenia; and a measurement of blood platelet levels may serve to determine if the presence of anti-platelet antibodies is causing a reduction in platelet number.

### V. Kits for Treating and Testing Autoimmune Disease Patients

The present invention provides kits for treating a primary autoimmune disease such as multiple sclerosis. A kit of this invention can contain, for example, a lymphocyte depleting drug (e.g., alemtuzumab), and a written instruction for informing a patient or a healthy care provider of contraindications of the drug, for example, the potential for an increased risk of developing a secondary autoimmune disease following treatment with the drug. The increased risk can be associated with or indicated by (i) a reduced fraction of platelet lineage cells (PLCs) among total cells, (ii) an increased IPF value, and/or (iii) an increased fraction of immature PLCs among the total PLC population, in any combination, and optionally (iv) increased antibodies against mature or activated platelets, in a biological (e.g., blood) sample from the patient as compared to a control subject.

In other embodiments, the invention provides kits for detecting the fraction of PLCs among total blood cells, the IPF value, and/or the fraction of immature PLCs among total PLCs, in a biological (e.g., blood) sample from an autoimmune disease patient, and/or for identifying patients at increased risk of developing a secondary autoimmune disease following lymphocyte depletion. Such kits can comprise reagents for detecting PLC markers such as CD41, CD61, SPARC, and/or TREML1 (and/or any other PLC/mature PLC markers described herein); immature PLC markers such as PDGFA, PDCD10, DAB2, RGS10, RGS18, and/or TSC22D1 (and/or any other immature PLC markers described herein); and/or IPF value; and optionally an instruction directing a user to take a biological sample (e.g., a blood sample) from a patient. Such kits will have been validated or approved by an appropriate regulatory authority for making medical diagnosis in patients, such as MS patients.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of immunology, medicine, medicinal and pharmaceutical chemistry, and cell biology described herein are those well-known and commonly used in the art. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Determination of Immune Cell Type Composition in Alemtuzumab-Treated Patients

### Methods:

### Clinical Trial and Sample Collection

Cryopreserved PBMC samples were obtained from the CAMM323 study (CARE-MS I, Clinicaltrials.gov identifier NCT00530348). In this study, patients diagnosed with relapsing-remitting multiple sclerosis (RR-MS), and who had not been previously treated with an MS disease-modifying therapy, were treated with alemtuzumab (12 mg/day, IV) for 5 consecutive days at baseline (TO) and for 3 consecutive days 12 months later (T12), or with subcutaneous interferon beta-1a (44 μg, thrice weekly). Whole blood (6-8 mL) was collected in CPT™ tubes with sodium citrate at T0, T12 (12 months post first course), and T24 timepoints (12 months post second course). This study analyzed samples from T0 and T24 time points for a total of 32 patients of which 11 developed secondary autoimmunity (sAI, defined by occurrence of thyroid events) following alemtuzumab treatment, 18 did not (non-sAI), and 3 patients had laboratory abnormalities (LA) as defined by the presence of autoantibodies (Jones et al., J Clin Invest. (2009) 119:2052-61). In the adjudication, a thyroid event was defined as either having a laboratory finding (e.g., abnormal TSH) or a clinical adverse event (AE). If diagnosed, the clinical AE was classified as Grave's disease (i.e., hyperthyroidism), Hashimoto's disease (i.e., hypothyroidism), transient thyroiditis, Grave's disease switching to hypothyroidism, Hashimoto's disease switching to hyperthyroidism, or uncertain. No demographic co-variate such as age, sex, or BMI showed associations with sAI (data not shown). The timeline for autoimmunity development is described in previous studies (Berger et al., CNS Drugs (2017) 31:33-50; Havrdova et al., Neurology (2017) 89:1107-16).

### PBMC Processing and Storage

Following blood collection, the CPT™ tubes were centrifuged at the clinical site, such that the red blood cells (RBCs) were captured within the gel barrier. The plasma layer and white buffy coat of PBMCs were mixed together prior to shipment. The CPT™ tubes were shipped to the laboratory at room temperature and processed within 60 hours of collection. PBMC collection was carried out in a class II biological safety cabinet. Cells were inverted into the plasma gently 5-10 times, then the CPT™ tubes were opened and the entire suspension above the gel was transferred into a sterile 15 mL conical tube. The volume of the solution was noted. After centrifugation of the samples at 300 g for 10-15 minutes, plasma was removed and discarded without disturbing the cell pellet. The pellet was resuspended by gentle pipetting, and Dulbecco's PBS (1×) was added to make up the volume to 10-13 mL. Samples were then centrifuged for 10-15 minutes at 300 g. The supernatant was aspirated without disturbing the pellet, and Dulbecco's PBS (1×) was added to bring volume to 10 mL. Samples were inverted and mixed gently. White blood cell count, and % lymphocyte and % monocyte counts, were determined using a Gen-S hematology analyzer. Cell viability was determined by staining with propidium iodide and processing on a FACSCalibur™. Subsequently, the samples were centrifuged for 10-15 minutes at 300 g and the supernatant was aspirated. The pellet was gently resuspended by pipetting. 2 mL of freezing solution Cryostor® CS10 (Stemcell Technologies, Cat #07930) was added and mixed using a 1 mL pipet. 1 mL of cell solution was aliquoted into 2 cryovials and stored at −80° C. for a minimum of 24 hours and a maximum of 72 hours before long-term storage in liquid nitrogen.

### PBMC Thawing and Pre-Processing for Single-Cell Workflow

As described previously in Hanamsagar et al., Sci Rep. (2020) 10:2219, cryopreserved PBMCs were thawed (2 vials at a time) in a 37° C. water bath for 1-2 minutes until a small crystal remained. The cryovial was removed from the water bath and the cell solution was transferred to a sterile 2 mL Eppendorf® tube using a wide bore pipet tip. The cryovial was washed with 1 mL of 0.04% BSA/PBS and the solution was transferred to the Eppendorf® tube. The sample was centrifuged at 150 g, 5 min, at room temperature (RT). The supernatant was carefully removed, and the sample was washed with 1 mL of 0.04% BSA/PBS using a wide bore pipet tip. The sample was washed twice more using the same conditions above, for a total of 3 washes. After the final wash, cells were resuspended in 1 mL of 0.04% BSA/PBS and counted using a hemacytometer (C-Chip™, SKC, Cat #DHCF015) with trypan blue (0.4%, GIBCO™ Cat #15250061) as the stain. If the viability was found to be lower than 75%, the sample was subjected to a “clean-up” step using Dead Cell Removal kit (Miltenyi Biotec, Catalog #130-090-101). Cells were washed again and resuspended in 500 μL of 0.04% BSA/PBS and counted. The volume was adjusted to 1× 10⁶ cells/mL of 0.04% BSA/PBS. Cells were run through a 10× Genomics Chromium device for encapsulation.

### Single-Cell Transcriptomics

After the cell volume was adjusted to 1×10⁶ cells/mL, the protocol for 10× Genomics 5′ gene expression library preparation was used. 8000 cells were targeted per sample. The quality of uniquely-indexed libraries was determined using a 2100 Bioanalyzer instrument (Agilent) with a High Sensitivity DNA kit (Agilent, Catalog #5067-4626), and quantified using a Kapa™ library quantification kit (Kapa Biosystems, Catalog #KK4824-07960140001) on the QuantStudio™ 7 Flex Real-Time PCR system. The libraries were diluted in 10 mM Tris-HCl buffer (pH 8.0) and pooled in equimolar concentration (2 nM) for sequencing.

Sequencing was carried out on a NOVAseq™ 6000 System (Illumina), using a NOVAseq™ 6000 S2 reagent kit (300 cycles, Illumina Cat #20028314). Sequencing depth and cycle number was as per 10× Genomics recommendations: Read 1=26 cycles, i7 index=8 cycles, Read 2=98 cycles, and the aim was set at a sequencing depth of 35,000 reads per cell.

### Single-Cell Pipeline, Preprocessing and QC

Single cell analysis was carried out as described previously (Hanamsagar et al., Sci Rep. (2020) 10:2219). Briefly, following BCL conversion, FASTqs were processed through CellRanger™ version v2.1.1 for demultiplexing, alignment, filtering, barcode counting, UMI counting, and generation of gene x barcode matrices. Subsequently, an in-house built pre-processing pipeline was run for background removal of ambient RNA and filtering out empty barcodes as well as stress and mitochondrial genes. The resulting hdf5 files were then inputted into an internal single-cell visualization tool called SPRING (Weinreb, Wolock, & Klein, Bioinforma Oxf Engl. (2018) 34:1246-8). SPRING also allows for compressing/reducing the size of large single-cell datasets for fast visualization. An in-house developed tool was then used to automatically annotate cell-types and cell-subtypes, as well as to identify novel cell-types. After close scrutiny, bad quality samples were excluded from analysis. Briefly, CellRanger™ web summaries for each of the samples were evaluated. If the “cliff graph” was found to pass QC, sequencing depth was evaluated. For samples with low sequencing depth, the libraries were re-pooled to account for low-read samples and re-sequenced. Data from all sequencing runs were combined and re-run through CellRanger™ Web summaries were evaluated again and samples with low cell counts and bad “cliff graphs” were flagged. All samples were processed through the internal pipeline for decontamination and filtering (as described above) and visualized on the SPRING portal. Samples that clustered poorly on the SPRING layout or had fewer than 100 cells were flagged. Thus, samples that failed CellRanger™ QC and SPRING clustering were eliminated from analysis. Seven such samples were identified and eliminated. The resulting new dataset was labeled Lemtrada_SC (sample clean-up). As these samples belonged to different timepoints of different patients, another dataset was created which eliminated paired samples from patients in the Lemtrada_SC dataset. This new dataset was called Lemtrada_PC (patient-clean-up).

### Results:

### Changes in Relative Abundance of Cell Types Before and After Alemtuzumab Treatment

As observed previously (Baker et al., JAMA Neurol. (2017) 74:961), an increase in B cells and a decrease in T cells was detected at the post-treatment timepoint (12 months after the second course; T24) (data not shown).

Further, individual proportions of these cell types in each sample, as determined using single-cell RNA-sequencing (scRNA-seq), strongly correlated to the clinically recorded lymphocyte count. This showed that the scRNA-seq relative abundance measurement, as measured using the developed pipeline, provided a reliable view on cell count. Additionally, it offered a deeper resolution in terms of cell types than routinely used standard fluorescence-activated cell sorting (FACS). No differences were observed in total monocyte and NK cell numbers in either group (data not shown). NK cell abundance from single-cell data also strongly correlated with clinical values (data not shown), further strengthening the possibility of using scRNA-seq data as a proxy for clinical values.

B and T Cell Differences in sAI Vs. Non-sAI Patients Before and After Alemtuzumab Treatment

Consistent with previous findings (Baker et al., 2017; Evan et al., Expert Opin Biol Ther. (2018) 18:323-34), it was found that numbers of naïve B cells increased significantly, whereas CD4 and CD8 T cell numbers were reduced post treatment (data not shown). No differences were observed in B and T cell subtypes in sAI versus non-sAI patients (data not shown).

### Classification of Unknown Cell Types

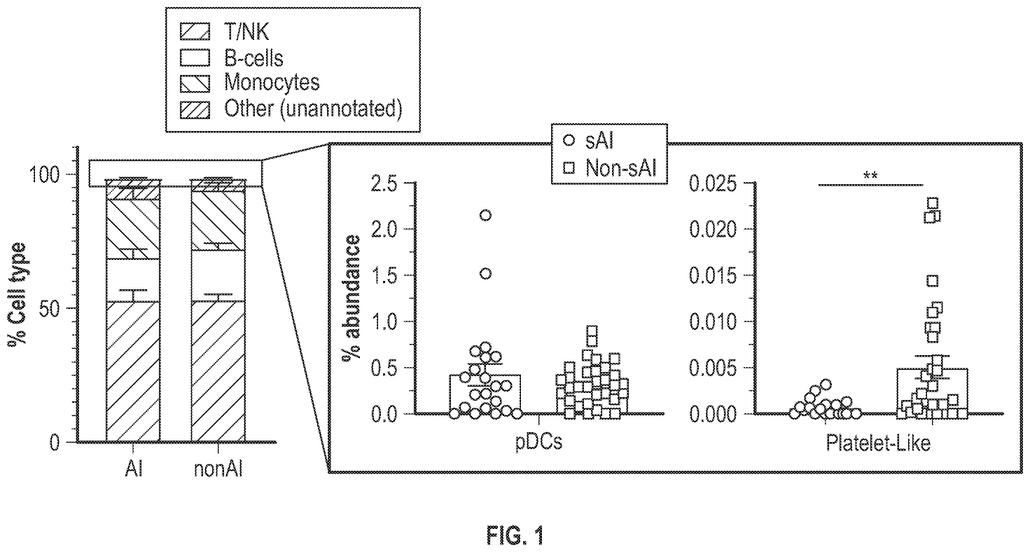

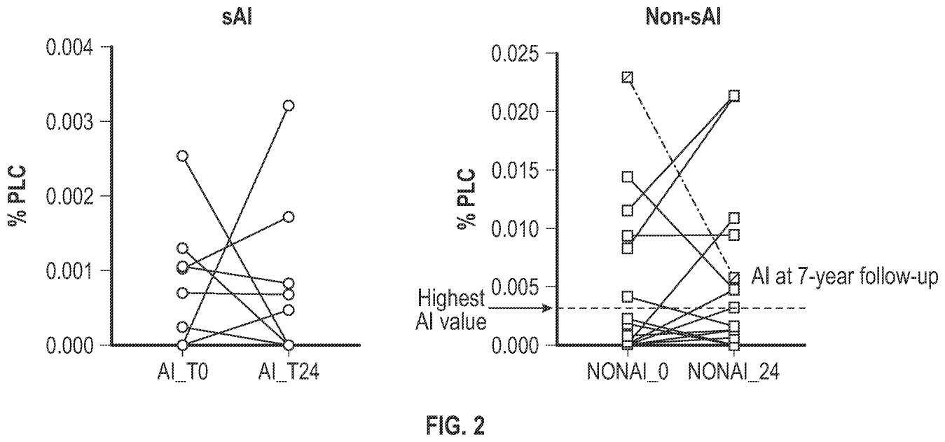

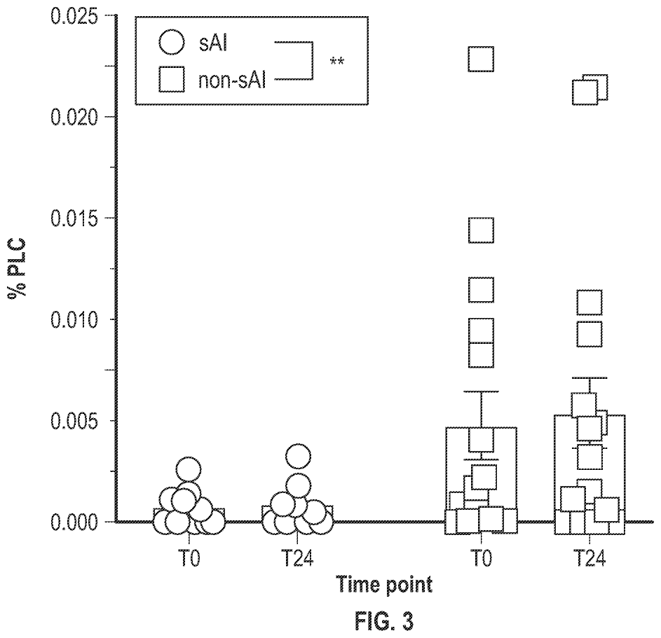

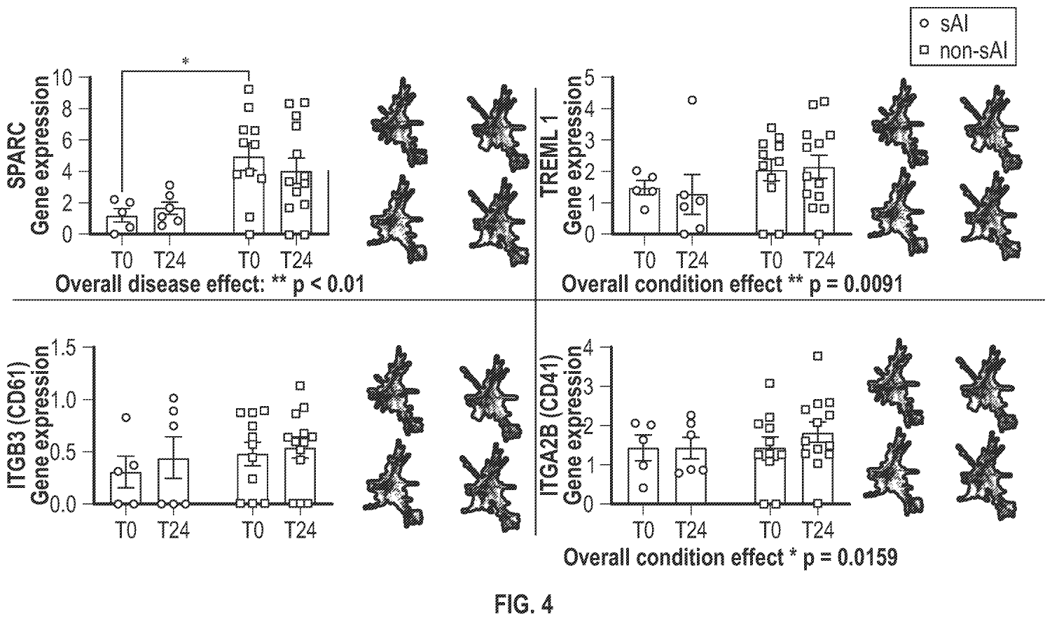

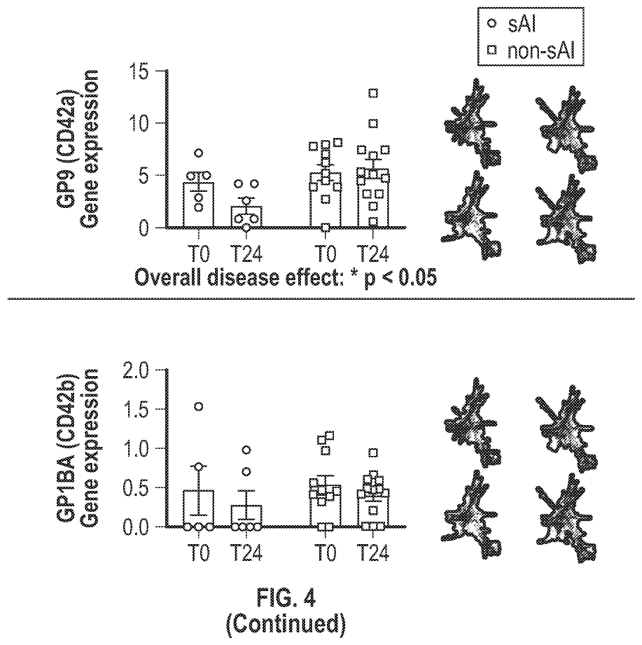

Since no difference in the relative abundance of known cell types or subtypes was observed between those with or without thyroid events, the abundance of cell types classified as unknown by the automated algorithm was analyzed. Surprisingly, this revealed a rare platelet-like cell-type (PLC) significantly lower in those with thyroid events compared to those without (FIG. 1, sAI 0.07%; non-sAI 0.52%). This effect was not driven by any particular patient (FIG. 2). The PLC percentage was low (<0.1%), but the difference between sAI and non-sAI was highly significant and held both at the pre-treatment and post-treatment timepoint (FIG. 3). Based on marker expression (FIG. 4), PLCs strongly resembled platelets, but expressed two additional surface markers at high levels that are not ubiquitously associated with platelets: SPARC and TREML. Platelets are common contaminants in PBMC preparations (McFarland et al., Cytom Part J Int Soc Anal Cytol. (2006) 69:86-94), however they are small and not expected to contain RNA. Thus, this cell type was hypothesized to constitute platelet lineage cells (PLCs) with special physical characteristics (e.g., larger size and transcript content) amenable to sc-RNAseq capture.

### Example 2: Identification of Platelet Lineage Cells (PLCs) Using FACS

In order to confirm the identity of the PLCs, a FACS experiment was carried out using samples from two healthy donors to estimate the relative abundance of PLCs throughout sample isolation and processing steps.

### Methods:

### Flow Cytometry:

### Pre-Processing of Cells

Whole blood samples were obtained from two healthy subjects using Sanofi's internal Donor Research Program in Framingham, MA. 75-100 mL of blood was collected per donor into CPT™ tubes containing sodium citrate (BD Biosciences, Cat #362761). In the first experiment, PBMCs were isolated from the blood as described above. Following this, PBMCs were washed twice with 1×PBS and counted using propidium iodide/acridine orange stain (Nexcelom, Cat #CS2-0106-5ML) on the Cellaca™ MX (Nexcelom, model MX-SYS1). Half of the freshly collected PBMCs were stored in Cryostor™ CS10 (Stemcell technologies Cat #07930) and frozen at −80° C. for 24 hours followed by storage in liquid nitrogen. After one week, cells were thawed, counted, and processed for flow cytometry. The remaining half of fresh PBMCs were processed for antibody staining and flow analysis. In the second set of experiments, the healthy donors were recalled, and blood was collected as above. RBC lysis was done using ACK lysis buffer (Gibco™, A10492-01, lot #2048611) as per manufacturer's instructions. Briefly, two 50 mL Falcon™ conical tubes were used for each donor. Approximately 10-12 mL of whole blood was poured into each 50 mL tube after initial centrifugation to remove plasma. ACK buffer was added to 45 mL and incubations were on a VWR variable speed rocker for 10 minutes. After a third 10-minute incubation, the cell pellets were mostly white, indicating red cells had been lysed and removed by washing. After this, the cells were processed for staining and flow cytometry.

### Compensation, Staining, and Flow Analysis:

Before running samples on the BD Influx™ (Becton Dickinson Influx Configurable, model 646500, s/n X64650000137), auto-compensation was done according to the Influx protocol. Briefly, two drops of compensation beads (see below) and one test of stain were mixed, incubated for 20 minutes on ice in the dark and then resuspended in 350 μL stain buffer in 5 mL Falcon tubes.

All cells were centrifuged at 300 g for 5 minutes at 4° C. Pellets were resuspended in 3 mL BD stain buffer, and transferred to a 5 mL Falcon polypropylene tube. Cells were centrifuged again, then the pellet was resuspended in 100 μL of staining panel and incubated on ice in the dark for 30 min. The staining panel consisted of: CD61-BV510 (5 μL/test), CD41A-APC (20 μL/test), TREML/TLT-1-FITC (5 μL/test), and SPARC-(350) (5 μL/test). After incubation, 3 mL of stain buffer was added and centrifuged. The supernatant was removed and pellets were resuspended in 2 mL of stain buffer and analyzed on the BD Influx as per the manufacturer's instructions. BD Influx™ information: Amplitude was set at 4.91, Drop Frequency was 44.70, stream focus was 15, Drop Position was 200, Max Drop was 101, Drop Delay was 28.43, and stream deflections for tubes were −84, −33, 33, 86.

| | | Catalog | Lot |
|---|---|---|---|
| Name | Supplier | Number | Number |
| Stain Buffer | Becton Dickerson | 554656 | 8145755 |
| Compensation Beads | Invitrogen ™ | 01-2222-42 | 2037700 |
| Sphero ultra-rainbow | Spherotech ™ | URFP-30-2 | AK02 |
| fluorescent particles | | | |
| FACS Accudrop beads | Becton Dickerson | 345249 | 822690 |
| PBS | Gibco ™ | 14190 | 2004029 |
| CD41A-APC | Becton Dickerson | 559777 | 8332624 |
| CD61-BV510 | Becton Dickerson | 563303 | 9126835 |
| TREML/TLT-1-FITC | R&D Systems | FAB2394G | AFEQ0118111 |
| SPARC (350) | R&D Systems | IC941U- | 15546582 |
| | | 100ug | |

### Gating Strategy:

Live cells were gated from the log scale on FSC and SSC, excluding dead cells. Of the live cells, “small cells” were gated using log scale on FSC and SSC because of their small size (2-3 μm). Singlets were gated off the FSC and SSC gate using Trigger-pulse width. CD41A⁺ and CD61⁺ were considered markers for platelets, and were gated off the singlet gate. From the CD41A⁺ CD61⁺ gate, PLCs were identified by being double-positive for SPARC and TREML1.

### Statistical Analysis

FlowJo™ (version 10) was used for analyzing flow data. GraphPad Prism (version 8) was used for generating graphs and performing statistical analyses. Levels of significance are indicated by: ***p<0.001, **p<0.01, and *p<0.05. PLC abundance results were tested for robustness against the presence of anti-PLGY antibody by excluding the single sAI patient (10553163), who also presented with this lab abnormality. Significance level was p<0.015 with this patient excluded.

### Results:

### PLC Identification

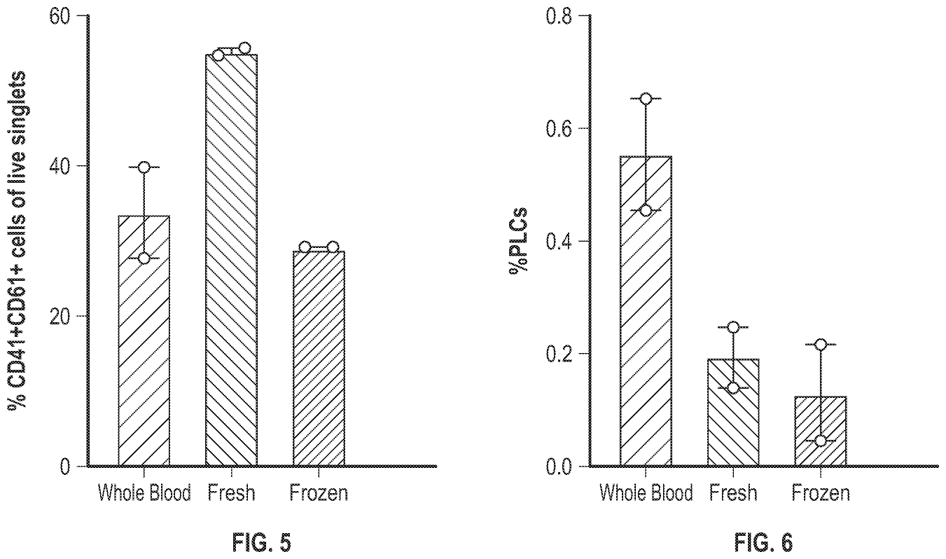

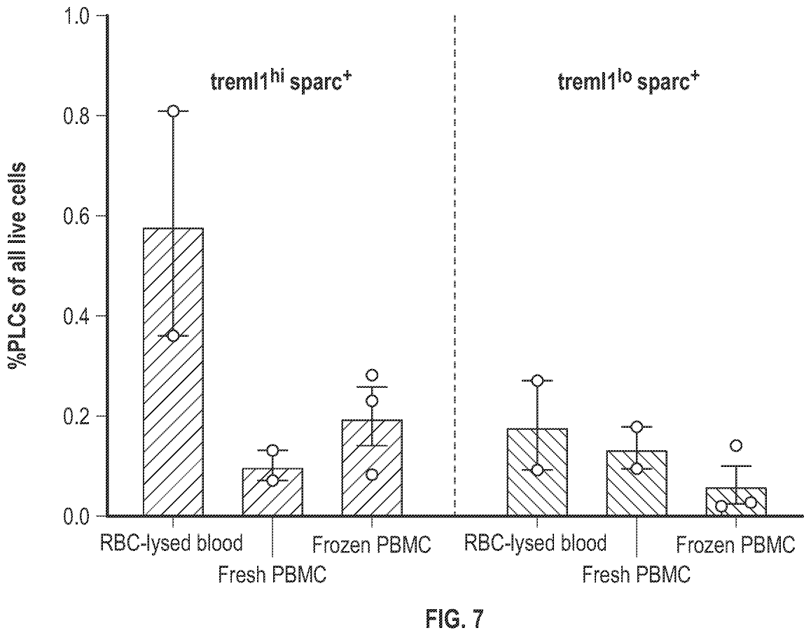

It was observed that the proportion of CD41⁺CD61⁺ cells was 35% in whole blood, increased to 55% in fresh PBMCs (after removal of neutrophils), and decreased to 30% in frozen PBMCs (FIG. 5). However, PLCs (defined as CD41 CD61″SPARC″TREML1″) constituted a mere 0.55% of whole blood and 0.1-0.2% of fresh and frozen PBMCs (FIGS. 6 and 7). To better understand the physical characteristics of PLCs in terms of size and granularity, they were analyzed with SSC/FSC gating, which showed that they appeared to be larger and more granular when compared to SPARC-TREML1⁻ (double-negative) platelets (data not shown).

### Difference in PLCs Between sAI Vs Non-sAI Patients Before Alemtuzumab Treatment

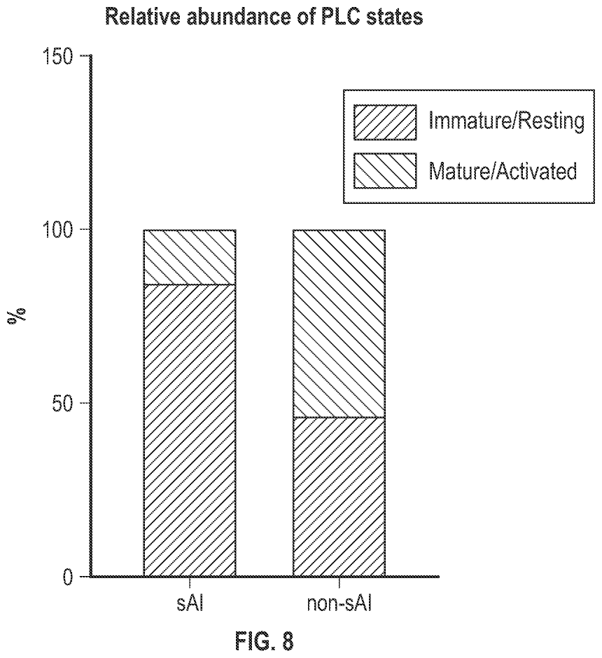

Having elucidated the identity of this novel cell-type, clustering of PLC transcriptomes was performed to determine if there were any qualitative differences in PLCs prior to alemtuzumab treatment between patients with thyroid events. It was found that there were distinct subsets of PLCs which differed in their expression of several markers and in their relative proportions (data not shown). Subset 1 (“immature/resting PLCs”) encompasses most PLCs in sAI patients, represented by five patients. It is characterized by lower expression of platelet markers, and higher expression of PDGFA, inhibitory markers (PDCD10), and nuclear proteins (DAB2, RGS10, RGS18, and TSC22D1). The second subset (Subset 2, “mature/activated” PLCs) is relatively higher in the actin genes ACTB and ACTG1, growth factor, a potent chemoattractant, and activator of neutrophils PPBP. This subset was also enriched in SPARC and TREML1 gene expression. These results suggest a difference in maturity and activation state between the two subsets, with the subset depicting immaturity/resting state of PLCs being enriched in those with thyroid events (FIG. 8).

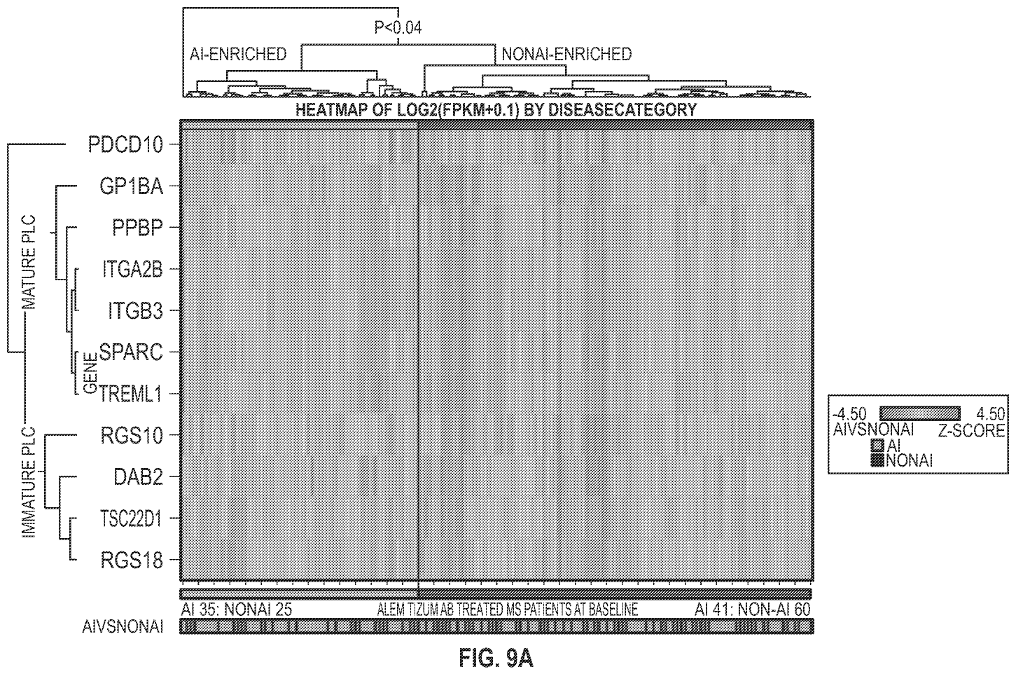

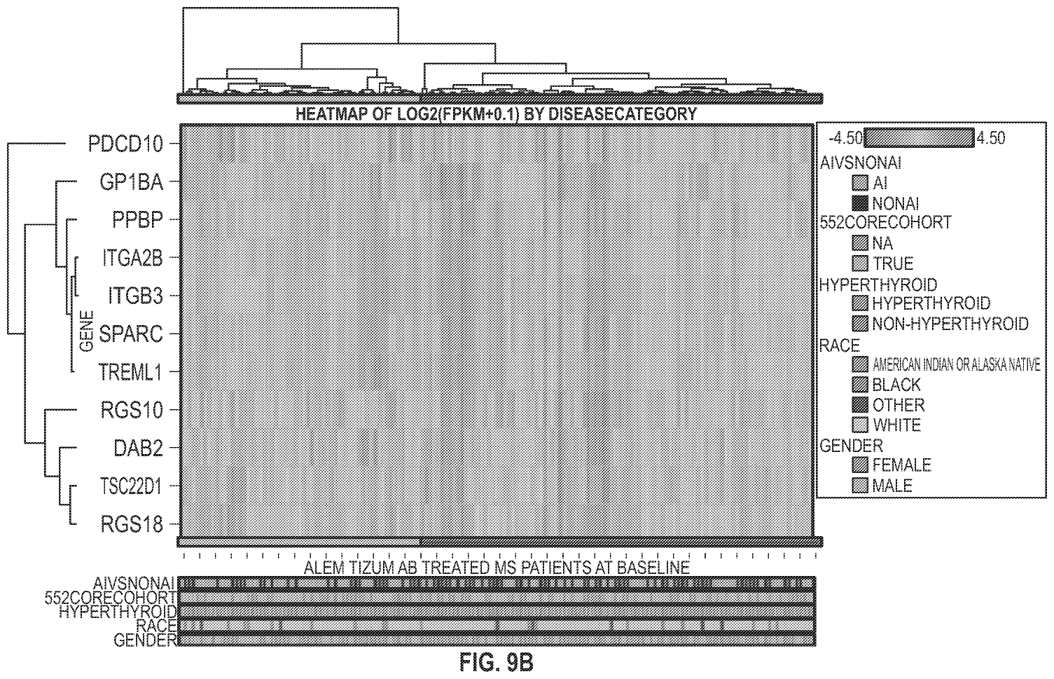

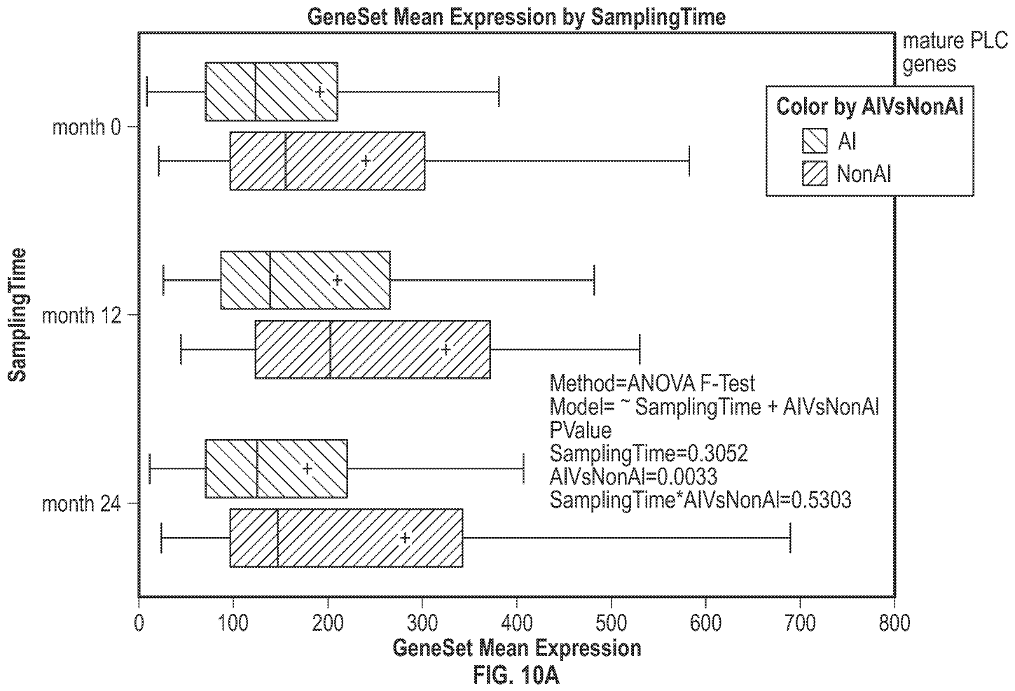

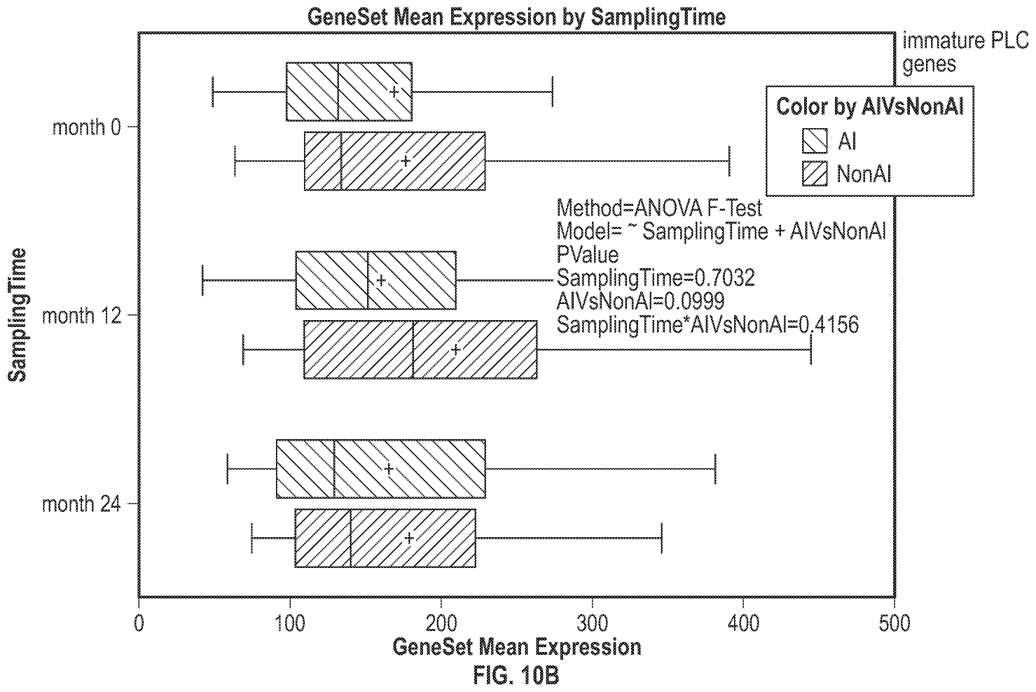

Indeed, an unsupervised clustering analysis of scRNA-seq data from 161 baseline patient samples revealed that patients who did not develop secondary autoimmunity had higher relative expression levels of six genes (GPIBA, PPBP, ITGA2B, ITGB3, SPARC, and TREML1) that may be markers for mature PLCs (FIG. 9A). This finding was independent of patient traits such as thyroid activity, race, and gender (FIG. 9B). The expression levels of the six mature PLC genes (FIG. 10A) and five immature PLC genes (PDCD10, RGS10, DAB2, TSC22D1, and RGS18) (FIG. 10B) were assessed at 0, 12, and 24 months after alemtuzumab treatment. Expression levels of the mature PLC genes were significantly reduced in patients that developed secondary autoimmunity. Expression levels of the immature PLC genes were about the same or slightly reduced in patients that developed secondary autoimmunity.

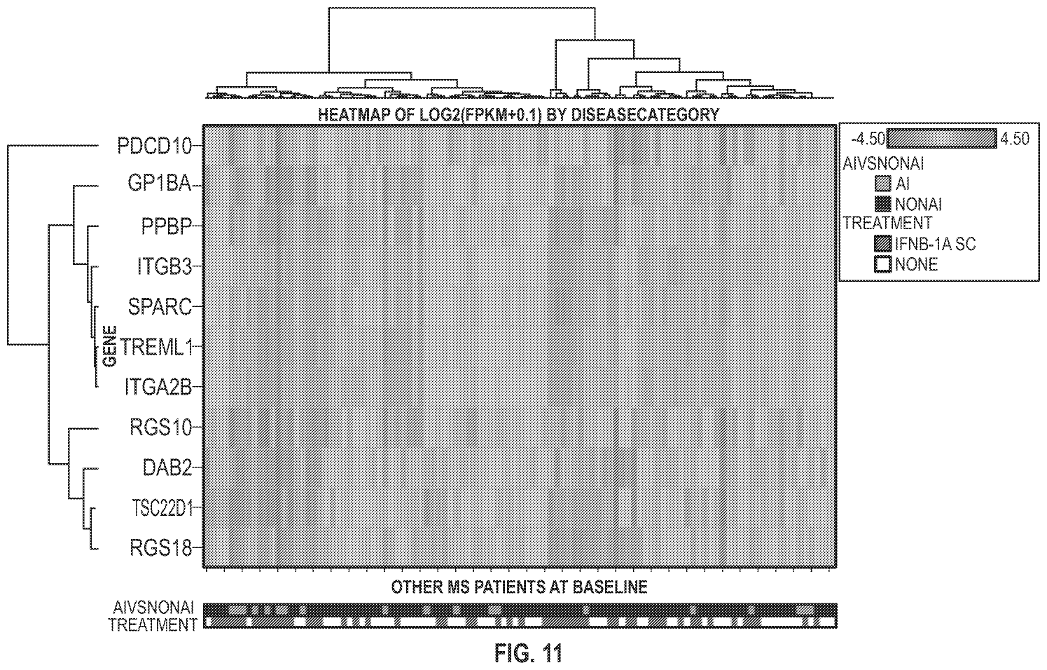

Similar unsupervised clustering analysis of scRNA-seq data from samples obtained from MS patients treated with IFN beta-1a revealed a different clustering pattern for the same mature and immature PLC genes, suggesting that these are specifically markers for the risk of developing secondary autoimmunity after alemtuzumab treatment (FIG. 11).

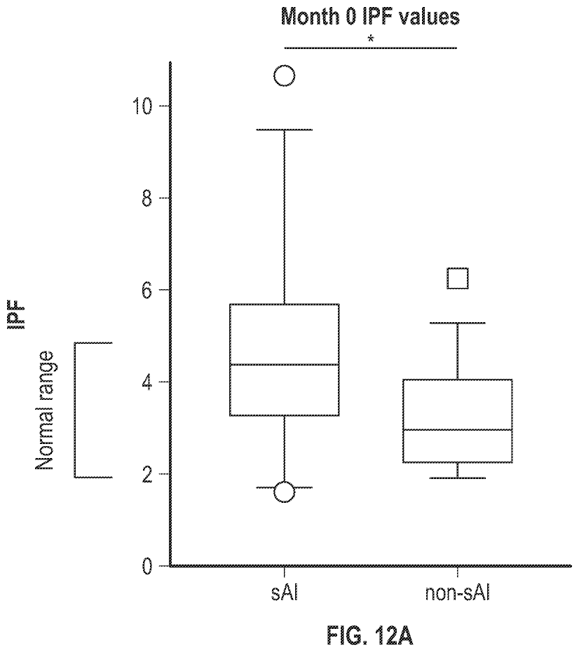

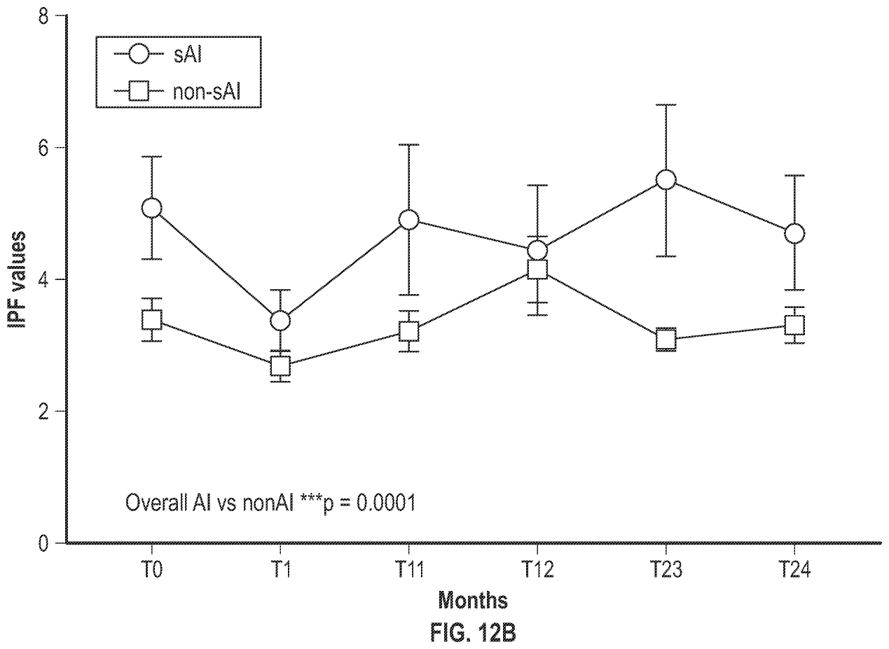

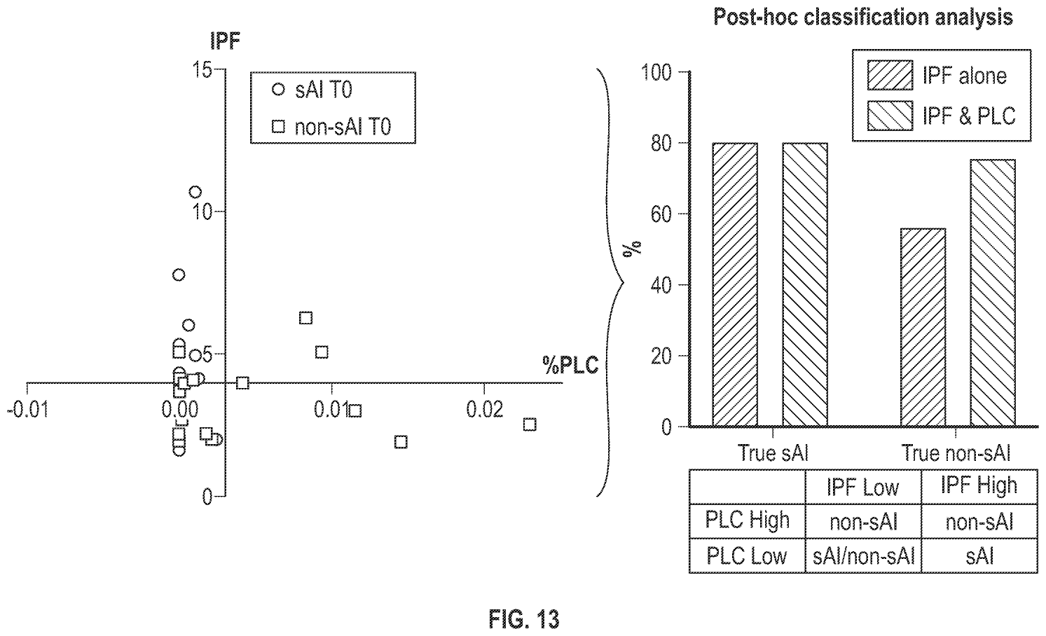

These studies suggest a difference in clinical measures of platelet maturity in patients with or without thyroid events after alemtuzumab treatment. To test this hypothesis, an analysis of Immature Platelet Fraction (IPF) data available for the cohort was carried out and it was found that patients with thyroid events had a significantly increased IPF at TO compared to those without (FIG. 12A). Moreover, patients with thyroid events tracked higher in IPF in monthly measurements taken throughout the two-year period (FIG. 12B). High IPF at T0 is not specific to sAI. However low relative PLCs and high IPF together appear to be a more specific indicator than high IPF alone (FIG. 13).

To investigate how PLC deficit relates to subclinical manifestations of sAI, data were generated from three additional patients in the cohort who did not present with clinical sAI events but had detectable autoantibodies: two had anti-PLGLY antibodies, the other anti-TPO antibodies. PLCs were absent in all three samples (data not shown).

Next, it was examined whether the PLC numbers at T0 could be indicative of AI outcome beyond the 4-year horizon. 7-year follow-up data were obtained for the same patients. It was found that the 7-year data was largely consistent with the 4-year data with the exception of a single patient who developed AI sometime after the 4-year follow-up. It was observed that this patient had drastically lower PLCs at T24 compared to T0 (FIG. 2).

What is claimed is:

1. A method for assessing the risk of developing secondary autoimmunity in a patient with a primary autoimmune disease following lymphocyte depleting therapy, comprising:
a) providing a blood sample from the patient; and
b) determining
(i) the fraction of platelet lineage cells (PLCs) among the total cells in the blood sample, wherein a reduced fraction of PLCs compared to a first reference is indicative of a heightened risk of developing secondary autoimmunity in the patient after treatment, and/or
(ii) the Immature Platelet Fraction (IPF) in the blood sample, wherein an increased IPF compared to a second reference is indicative of a heightened risk of developing secondary autoimmunity in the patient after treatment.

2. A method for treating a patient with a primary autoimmune disease, comprising:
a) selecting a patient who has been diagnosed as not being at a heightened risk of developing secondary autoimmunity after lymphocyte depleting therapy, wherein the risk has been diagnosed by determining
(i) the fraction of platelet lineage cells (PLCs) among the total cells in a blood sample from the patient, wherein a reduced fraction of PLCs compared to a first reference is indicative of a heightened risk of developing secondary autoimmunity in the patient after treatment, and/or
(ii) the Immature Platelet Fraction (IPF) in the blood sample, wherein an increased IPF compared to a second reference is indicative of a heightened risk of developing secondary autoimmunity in the patient after treatment; and
b) administering a therapeutically effective amount of the lymphocyte depleting therapy to the patient.

3. The method of claim 1 or 2, wherein said determining step comprises determining both (i) and (ii).

4. The method of any one of claims 1-3, wherein the primary autoimmune disease is multiple sclerosis (MS).

5. The method of any one of claims 1-4, wherein the lymphocyte depleting therapy is a lymphocyte depleting antibody therapy.

6. The method of claim 5, wherein the antibody is an anti-CD52 antibody or an antigen-binding portion thereof.

7. The method of claim 6, wherein the anti-CD52 antibody has the six CDRs of alemtuzumab.

8. The method of claim 6, wherein the anti-CD52 antibody has the heavy and light chain variable domains of alemtuzumab.

9. The method of claim 6, wherein the anti-CD52 antibody is alemtuzumab.

10. The method of any one of claims 1-9, wherein the first and second references are obtained from a patient with said primary autoimmune disease who does not develop secondary autoimmunity after lymphocyte depleting treatment, or from a healthy subject.

11. The method of any one of claims 1-10, wherein the blood sample is an erythrocyte-lysed blood sample.

12. The method of any one of claims 1-10, wherein the blood sample is a peripheral blood monocyte cell (PBMC) sample, optionally wherein neutrophils in the sample have been removed.

13. The method of any one of the preceding claims, wherein the PLC fraction is reduced by >2 standard deviations compared to that of a control subject.

14. The method of any one of the preceding claims, wherein the IPF value is increased by >2 standard deviations compared to that of a control subject.

15. The method of any one of the preceding claims, wherein the PLCs are characterized by being CD41⁺CD61⁺SPARC⁺TREML1⁺.

16. The method of any one of the preceding claims, further comprising the step of determining the fraction of immature PLCs among the total PLC population in the biological sample from the patient, wherein an increased fraction of immature PLCs compared to a third reference is indicative of a heightened risk of developing secondary autoimmunity in the patient after treatment.

17. The method of claim 16, wherein the immature PLCs are characterized by being CD41^{low}CD61^{low}PDGF A^{high}PDCD10^{high}, optionally further by being DAB2^{high}RGS10^{high}RGS18^{high}TSC22D1^{high}.

18. The method of claim 16 or 17, wherein the third reference is obtained from a patient with said primary autoimmune disease who does not develop secondary autoimmunity after lymphocyte depleting treatment, or from a healthy subject.

19. The method of any one of the preceding claims, wherein the secondary autoimmunity is selected from the group consisting of immune thrombocytopeniaurpura (ITP), Graves' disease, Hashimoto's disease, Goodpasture's disease, membranous glomerulonephritis, red cell aplasia, autoimmune thyroid disease, transient thyroiditis, autoimmune hemolytic anemia, diabetes mellitus type 1, alopecia areata/alopecia totalis, vitiligo, myalgia, sarcoidosis, autoimmune neutropenia, autoimmune hepatitis, and autoimmune lymphopenia.

20. The method of any one of the preceding claims, wherein the primary autoimmune disease is relapsing MS.

21. The method of any one of the preceding claims, wherein the primary autoimmune disease is relapsing-remitting multiple sclerosis (RR-MS).

22. The method of any one of the preceding claims, wherein the primary autoimmune disease is secondary progressive MS (SPMS).

23. A lymphocyte-depleting therapy for use in treating a primary autoimmune disease in a patient, wherein the patient has been selected as not being at a heightened risk of developing secondary autoimmunity after the lymphocyte depleting therapy, wherein the risk has been diagnosed by determining
(i) the fraction of platelet lineage cells (PLCs) among the total cells in a blood sample from the patient, wherein a reduced fraction of PLCs compared to a first reference is indicative of a heightened risk of developing secondary autoimmunity in the patient after treatment, and/or
(ii) the Immature Platelet Fraction (IPF) in the blood sample, wherein an increased IPF compared to a second reference is indicative of a heightened risk of developing secondary autoimmunity in the patient after treatment.

24. Use of a lymphocyte-depleting therapy in the manufacture of a medicament for treating a primary autoimmune disease in a patient, wherein the patient has been selected as not being at a heightened risk of developing secondary autoimmunity after the lymphocyte depleting therapy, wherein the risk has been diagnosed by determining
(i) the fraction of platelet lineage cells (PLCs) among the total cells in a blood sample from the patient, wherein a reduced fraction of PLCs compared to a first reference is indicative of a heightened risk of developing secondary autoimmunity in the patient after treatment, and/or
(ii) the Immature Platelet Fraction (IPF) in the blood sample, wherein an increased IPF compared to a second reference is indicative of a heightened risk of developing secondary autoimmunity in the patient after treatment.

25. The lymphocyte-depleting therapy for use of claim 23 or the use of claim 24, wherein the treatment and/or the selection is in accordance with the method of any one of claims 1-22.
